(19) United States
(12) Patent Application Publication
(10) Pub. No.: US 2011/0070617 A1
(43) Pub. Date: Mar. 24, 2011

(54) Liquid methanol fuel production from methane gas at bio-normal temperatures and presure

(76) Inventor: Mathew M. Zuckerman, Toluca Lake, CA (US)

(21) Appl. No.: 12/586,659

(22) Filed: Sep. 24, 2009

(51) Int. Cl. C12P 7/04

(52) U.S. Cl. 435/157

(57) ABSTRACT

Through staged and monitored control of gas, liquid, and solid source materials, the highly-efficient enzymatic ‘natural factory’ of specific methanotropic bacteria, which evolved dual, alternative, metabolic channels, can be manipulated for human goals. The first stage sets these bacteria to producing liquid methanol by oxidation of methane gas under aerobic conditions (their high-energy channel), which is harvested at the peak. The second stage, by establishing anaerobic conditions and providing supplementary metals, forces the bacteria to use their lower-energy channel for inorganic hydrogen-donor to organic-energy-transport, during which the older and weaker organisms become ‘food’ for newer and (relatively) stronger organisms. This accomplishes the desired result of liquid methanol production without employing a human-engineered industrial-chemical process with the costly high energy requirements associated with temperatures and pressures required by the prior art for converting methane gas to liquid methanol.

### REFERENCES

Combustion, 4^{th} Edition, Elsevier, Inc, 2008, p 709.

Handbook of Applied Chemistry, Hemisphere Publishing, 1983, ppIV/3 8-9

Baik, M-H., Newcomb, M., Friesner, R. A., et. Al., Mechanistic Studies on Hydroxylation of Methane by Methane Monoxygenase, Chemical Reviews, 2003, vol. 103, p. 2385

Crabtree, R. H., Aspects of Methane Chemistry, Chemical Reviews, 1995, vol. 95, p. 987.

Niedzielski, J. J., Schram, R. M., Phelps, T. J., Herbes, S. E., and White, D. C., A Total-recycle-expanded-bed Bioreactor design which allows direct headspace sampling of volatile chlorinated aliphatic compounds, J. Micro. Meth. 10:215-223 (1998).

Wikipedia on NAD+/NADH: http://en.wikipedia.org/wiki/NADH#cite_note-Pollak-0.

### BACKGROUND OF THE INVENTION

Our world is generally agreed as being near Hubbert's Peak, the point where one-half of all of the crude oil (petroleum) that can ever be obtained has been produced. Yet we are still refining ⅔ of this exhaustible resource into gasoline and diesel fuel. The other ⅓ is made into lubricants, plastics and other products that are a significant part of the underpinning of the western way of life. Crude oil as a base of these products is hard to replace: a reasonably foresighted nation might well soon decide that crude oil is too valuable to continue to be burned as a fuel.

Natural gas is found both in many of the same areas as crude oil, and in reserves more abundant than crude oil. Methane gas (CH₄) is the principal component by volume of natural gas (75% v/v). Methane gas also arises from the natural biodegradation of organic materials. Methane gas both forms the majority (60% plus) component produced when organic materials are composted, and is the largest component arising from the anaerobic decomposition of wastewater sludges. Methane gas has well-known qualities and is most easily used in a liquid form, i.e. as methanol. Methanol burns in air forming carbon dioxide and water using this reaction:

2CH3OH+3O2→2CO2+4H2O.

A principal object of the present invention is to produce and liquefy methanol from methane gas to enable the methanol to be used as an abundantly available liquid fuel, that is economically and easily transportable to supplement or replace crude oil based liquid fuels such as gasoline.

Today, carbon footprint and global warming are topics of concern. The combustion of carbon is a major contributor to the problem. Hydrogen is a far better fuel. Hydrogen, with an atomic weight 1/12 that of carbon, produces twice the energy per molecule compared to carbon. Historically, fuels were carbon based as wood, peat and coal where the fuels of choice. The recent transition to liquid hydrocarbon fuels is changing this and has, over the decades, been shifting the content of our average fuel away from carbon towards hydrogen, particularly in the advanced or developing countries. However, the recent increase in demand for electrical power in the emerging economies of India and China has reversed this process; for these nations together are reported to be commissioning a new coal-fired electric power generating plant at a rate of one per week.

Methanol as a fuel has a 4:1 ratio of hydrogen to carbon which is higher than ethanol with a 3:1 ratio and gasoline, measured as octane, with a 2.25:1 ratio. The world needs, for its survival, to move quickly to using methanol as a fuel in order to reestablish the desirable trend of replacing carbon with hydrogen as its prime fuel source. By subsisting methanol for other liquid fuels we can meet this need, and by making methanol production feasible at normal temperatures and pressure (thus reducing the need for energy in the processing) we can even further reduce the carbon-fuel pressure on our global environment.

Originally the product of a destructive distillation of wood (which is why methanol is sometimes called ‘wood alcohol’), methanol is currently produced in volume by an “industrial-chemical process”. Most industrially-produced methanol is made by hydrogenating carbon monoxide in the presence of a mixed catalyst (usually zinc oxide or chromium oxide). One such approach requires both pressurizing the CO gas to between 200 and 700 bars (92,900 to 10,150 lb/in²; hundreds of times greater than the 1.01325 bars of a ‘standard atmosphere’), and carrying out the hydrogenation at 318 degrees C. (716 degrees F.). An alternative that uses ‘only’ 10-20 atmospheres of pressure, 850° C. temperature, steam, and two stages of catalytic processing (requiring first a nickel, and then a combination copper, zinc oxide, and alumina catalyst), is described at http://en.wikipedia.org/wiki/Methanol. A third and very recent approach announced success with ‘low’ temperatures—only twice boiling heat (200° C.)—as well as a platinum-based catalyst; though it also apparently requires fuming sulfuric acid as an oxidant, thus posing ‘significant challenges regarding compatible materials and cost’, to say nothing about safety or environmental concerns. See Chemical & Engin'g News, Vol. 87 Iss. 35, Aug. 31, 2009, p. 7, or http://pubs.acs.org/cen/news/87/i35/8735notw2.html.

These processes each constitute an epitome of ‘industrial processing’. They all require advanced metallurgy, sophisticated piping and temperature controls; present notable risks from mechanical or system failure; require extensive maintenance and supervision—and involve significant energy capital cost, and operating costs including the energy to create the high temperatures and pressures required. Because of the high cost of methanol production, little is made in comparison to the demand for fuel. Currently less than 10% of the world's production of methanol is used as a fuel. Interestingly, the major use of methanol now is as a chemical feedstock for the production of formaldehyde, which is a metabolic byproduct of microbiological conversion of methane gas to liquid methanol. If the world's entire current production of methanol were converted to supplementing the gasoline demand of the US it would only satisfy 2% of that demand. There is a need for a more commercially viable process to process methane into methanol.

Nature, with a billion years and a world's variety of ecological niches, evolved a way to effect the transformation from methane to methanol at the lower-half of standard or liquid-water temperatures and pressures (between 10 and 45° C. and at ambient or one-bar atmospheric pressure). Certain methane-loving (methanotropic) bacteria, discovered in soil and semisolid environments (muds), evolved the ability to use methane gas as their sole carbon source by transforming it into methanol, which they then further process and consume. These bacteria (also known as ‘methanotrophs’) generally reduce the atmospheric load of hydrocarbons by oxidizing methane gas formed in biological systems such as muds. Such methanotropic bacteria are termed facultative for they can survive in both aerobic (oxygen containing) and anaerobic (no oxygen containing) environmental conditions. These bacteria are moderately temperature sensitive within the range of ‘normal’ environmental temperatures. Their metabolic pathway is shown in FIG. 1.

16s rDNA sequence analysis has clarified the classification of the phylogenetic relationships of these organisms into eight genera of Methanotrophs (Methanotrophic bacterium), which have been further classified into three groups. Type I, using the ribulose monophosphate pathway, assimilate formaldehyde produced from methane via methanol. Type II, using the serine pathway, also assimilate formaldehyde produced from methane. Type III use a combination of the pathways for Type I and Type II. There are five Type I, two Type II and one Type III bacterium. The Type I are Methylomonas, Methylmicrobium, Mthhylobacter, Methylocaldum, and Methylosphaera. The Type II are Methylocystis and Methylosinus. The type III is Methylococcus. The preferred embodiment of the present invention utilizes the Type I bacterium Methylomonas Methanica. However, the conditions in various locations throughout the world may dictate the presence of other supporting bacterium or even the dominance of another one of the eight types. Because the bacteria are ubiquitous in soils and muds it is only necessary to limit the carbon source to methane under aerobic conditions in a stoichometric ratio of methane to oxygen of 1:1 in an aqueous solution for a period of time which varies with the temperature, to develop sufficient Methanotrophic bacteria mass to accomplish the oxidation of methane to methanol.

### SUMMARY OF THE INVENTION

The present invention teaches how to produce methanol from methane through a process of selectively choosing the right bacteria, managing their environment (aerobic/anaerobic and batch/continuous conditions, timing/materials inputs and outputs); monitoring the sum of their internal aqueous/gaseous processes; and from specified inputs first create and then draw off the outputs of the latters' innate ‘natural factory’. Instead of insisting on a human-engineered, high-pressure and high-temperature and high cost, inorganic, and direct chemical transformation of the source material (methane gas) into methanol, intelligent design of the production environment, intelligent handling of the bacteria—that handling including identification, selection, monitoring, and resource-and-outputs manipulation—and intelligent and knowledgeable manipulation of the overall transformative processes—provides a far lower-cost and more-elegant solution than the direct chemical synthesis of the prior art.

Providing the correct (safe) atmospheric and temperature conditions, selectively introducing and removing the reactive chemical and biological inputs and outputs according to stoichometric valuations and operational measurements based on the bacterial selections and conditions, creates the desired mixture(s) of output chemicals which can then with reasonable economy produce the sought-after liquid product(s).

The present invention has the significant advantages of operating at ambient temperatures and pressure over current commercial processes for converting methane to methanol as the latter require high pressure, high temperatures and at least one metal catalyst. The present invention also presents significant economies over lignifying of natural gas (LNG), because it requires significantly lower capital and operating costs for each production facility, as well as a far cheaper, and safer, transportation and handling infrastructure; potentially only minor adaptations to the existing distribution networks. Still further advantages arise from the inherent efficiency advantage of biological over directly, human-engineered systems, as the biological activity of the properly selected and ‘nourished’ bacteria doubles for each ten degree increase in temperature in the normal range for bacteriological systems of 10 to 45° C. temperature; while the processing can be adapted to allow the use of brackish or polluted waters to operate in remote areas and even at desert conditions.

### DESCRIPTION OF THE DRAWINGS

FIG. 1 is known prior art and describes the life-cycle of a methanotropic bacterium, cycling through its assimilation of formaldehyde and formate produced from methane (via methanol in aerobic conditions) with the production of more bacteria and the respiration ‘by-product’ generated. Prior art does not speak to the availability of products for harvesting at each stage, for such harvesting was presumed to terminate the life-cycle and result in death of the system by depriving the bacteria of food.

FIG. 2 is also prior art, and is a diagram of the principal oxidation/reduction reactions, reactants and enzymatic systems that take place in methanotropic bacterial experiencing a cycle of aerobic and anaerobic environments.

FIG. 3 is a slightly expanded diagram of the first stage of methanotropic bacteria metabolic processing, in an aerobic environment.

FIG. 4 is a slightly expanded diagram of the second stage of methanotropic bacteria metabolic processing, in an anaerobic environment.

FIG. 5 is a diagram of the combined two-stage or full-cycle processing system for using piped-in methane gas, through timed and measured interruption and harvesting of intermediate (by-)products, through first the aerobic and then the anaerobic cycles of the methanotropic bacteria at normal pressures and temperatures, to produce and subsequently liquify methanol for commercial use. The first reaction tank [1] starts with a volume (in the preferred embodiment 10,000 gallons) of mixed liquor suspended solids (MLSS), which is 99.5% by weight water and 0.5% by weight a bacterial-mixed-liquor suspended solids concentration which is 90% by weight of the volatile fraction thereof a bacterial mass of Type I bacterium Methylomonas Methanica. This 10,000 gallon MLSS does not completely fill the first reaction tank leaving a gas/liquid interface surface [41]. Connecting to the first reaction tank are an intake for normal air [10] through an air compressor [11] and an air intake valve [12], and an intake from a methane gas source [13] through a methane compressor [14] and a methane intake valve [15]; and connected from the first reaction tank [1] above its interior gas/liquid interface surface [41] is a gas recirculating line running through an excess CO₂ scrubber [7] and an excess water vapor scrubber [8] that in turn connects to the combined gas intake for the first reaction tank, whereby both intake valves and recirculating line are controlled by a methane gas concentration monitor [16], so that the gaseous mixture actually introduced into the first reaction tank [1] will comprise methane to oxygen in a 1:4 ratio by volume. Once the first reaction tank has been filled with the MLSS the tank is sealed and gas containing the 1:4 volumetric mix of methane to oxygen is continuously introduced to the first reaction tank (in the preferred embodiment, by bubbling up from the bottom), then recirculated with excess CO₂ and water vapor (H₂O) scrubbed out. In the preferred embodiment this process continues for 4 hours at 20° C. for the stated volume of MLSS and bacterial-MLSS, with, in the preferred embodiment, the oxidation reduction potential (ORP) of the MLSS being continuously monitored by the ORP monitor [9], such that the recirculation of the gas stops when the ORP monitor [9] reads minus 0.5 to 5 millivolts. Then the MLSS is transferred to a clarifier [2] in the preferred embodiment designed such that in two hours with the stated volume for a settling rate of 0.5 gallons per minute per square foot of surface loading at 20° C., which also has a gas/liquid interface [42] but is sealed from the outside atmosphere. After those two hours, the liquid in the clarifier is run continuously through a thermal separator [4] which is kept at a temperature at or above the boiling point of methanol of 64.6° C. The liquid from the separator returns to the clarifier, while methanol that has been separated is run through a water-cooled condenser [5], and the resultant re-liquefied methanol [50] is prepared for transport. Next the MLSS remaining in the clarifier [2] is sent to a second reaction tank [3] similar to the first; and the liquid remaining in the thermal separator [4] is, supplemented along the way with an input of water and Ferric Oxide (Fe₂O₃) [6], also sent to the second reaction tank, again leaving in the second reaction tank a gas/liquid interface [43] and the second reaction tank is sealed against the outside atmosphere. Now an anaerobic environment is established for the MLSS and methanotropic bacteria, as the intake from the methane gas source [13] through the methane compressor [14] and a second methane intake valve [25], and a gas recirculating line running from above the gas/liquid interface [43] through through an excess CO₂ scrubber [27] and an excess water vapor scrubber [28] are both connected and controlled through a second methane gas concentration monitor [26] and let flow into the second reaction tank [3], both the second methane intake valve [25] and gas recirculating line being controlled by the second methane gas concentration monitor [26] such that the gaseous mixture actually introduced into the second reaction tank [3] will comprise as close as is practical to 100% methane. This 100% methane gas will continue to be recirculated through the second reaction tank [3], continuously introduced to the second reaction tank [3] (in the preferred embodiment, by bubbling up from the bottom), then recirculated with excess CO₂ and water vapor (H₂O) scrubbed out. In the preferred embodiment this process continues for 4 hours at 20° C. for the stated volume of MLSS and bacterial-MLSS, with, in the preferred embodiment, the oxidation reduction potential (ORP) of the MLSS being continuously monitored by a second ORP monitor [29], such that the recirculation of the gas stops when the ORP monitor [9] reads plus 1 to 6 millivolts. From this point, with the methanotropic bacterial mass, and their internal MMO-enzyme reset for aerobic oxidation and methanol production, the contents of the second reaction tank [3] are available to draw from and/or add to such amounts of MLSS and bacterial-MLSS, and reduce or add solids and/or water, as to form the proper starting MLSS ratio and restart the cycle.

FIG. 6 is a timing cycle diagram for production as described in FIG. 5 once start-up is complete and a commercial production cycle is established on a 24/7 schedule.

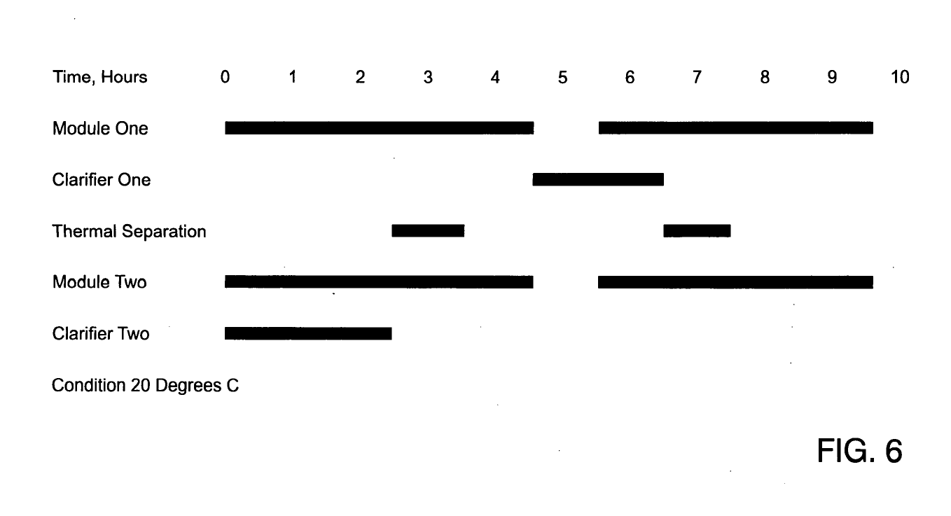

### DETAILED DESCRIPTION OF THE INVENTION

Methanotropic bacteria evolved an enzyme known as Monoxygenase (‘MMO’) that, depending on the type of bacteria, is either a dominant copper-containing or iron-containing organometallic complex. This MMO enzyme allows the methanotropic bacteria to oxidize methane as a carbon source at atmospheric pressure and in the lower temperature range of liquid water, 10° to 45° C. Eons of evolutionary selection hammered out a solution that hundreds of years of human scientific progress has not been able to duplicate; yet intelligent design, backed by insightful vision and careful research and carried out with comprehending supervision, now can turn these microorganisms' internal functionality to human benefit.

The present invention is a two-stage process that—by controlling and interrupting at the right points—uses these methanotropic bacteria's enymzatic processes. By controlling atmospheric composition—all without requiring extremes of either high or low pressure, or high or low temperature—and regulating the substances introduced to and withdrawn from the ‘reaction tank’, this invention allows a multi-stage exploitation of bacteria which in the everyday environment are seen as lacking any exploitably useful function.

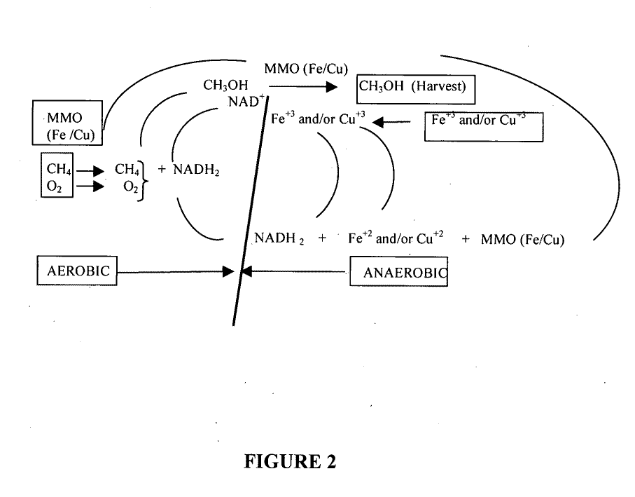

The two stages are broadly described in FIG. 2.

To better the understanding of the overall cycle of processing in this invention shown in FIG. 2, the practitioner experienced in the prior are is presumed to know both the energy-producing organelle of organisms, the mitochondria, and their chemical cycle for generating energy, namely the ‘Krebs cycle’. The present invention, looking to provide energy to the macro world, has looked to the power supply of the micro world for inspiration. Calcium and Magnesium are the inorganics that provide a key role in an energy-producing cycle like the mussel movement. In the present invention Iron and Cupper are the inorganics that provide a similar role.

In both the Krebs cycle and the present invention, NADH₂ and NAD⁺ play a key role in energy transfer. That is where the similarity ends. The Krebs cycle is far more complex and is designed to generate energy for the organism. In the present invention the energy source the methanotropic bacteria prepare for their use is harvested as liquid methanol for human use; and then the inorganics are inserted into the process in order to divert and reset the methanotropic bacteria's cycle through reduction of the NAD⁺ to NADH₂. Instead of following the natural full methanotropic bacterial cycle, the current invention short-circuits it at the most productive point for human needs, and then ‘resets’ the bacteria to the start of their natural cycle by establishing the lower-energy anaerobic condition and concurrently supplying inorganics. This directs the bacteria into a ‘carnivorous’ state where the stronger and younger bacteria consume the older and weaker as food while regenerating the NADH₂, with protons supplied by the conversion of the inorganic from the +3 to the +2 valance state, through augmenting the functioning of the bacteria's organo-metallic MMO enzyme by maintaining an excess of the metallic in solution so as not to limit production of MMO enzyme. The MMO enzyme makes oxidation of methane possible at ambient temperature and pressure. The second stage's resetting of the biological cycle can be further manipulated to produce additional bacterial biomass or decrease excess MLSS.

The present invention uses methane gas in two different modes during the first and second stages of the cycle. First the methane gas serves as the carbon source in an oxidation reaction used by the methanotropic bacteria under aerobic conditions; and secondly the methane gas serves as the environmental control for the methanotropic bacteria to block the use of oxygen as a proton acceptor for the oxidation reaction and force the resetting processing for the NAD⁺ from metallic salts, which the methanotropic bacteria evolved to cope with anaerobic conditions. Additionally, to avoid any risk of combustion or explosion, the first stage uses a methane-to-air mixture below the lower flammability limit of 5% weight for weight with oxygen; while the second state uses a methane-without-oxygen mixture which both blocks the natural cycle's further oxidative processing and combustion as it exceeds the upper flammability limit of 15% w/w with oxygen. (Combustion, 4^{th} Edition, Elsevier, Inc, 2008, p 709.). Methane gas is added and CO₂ and water vapor are removed from the monitored and controlled gas stream in both modes when recycling the gas as part of maintaining the current stage's mix.

A first reaction tank is mostly filled with a selected methanotropic bacteria in a mixed liquor suspended solids concentration (MLSS). In the preferred embodiment, the methanotropic bacteria will be the Type I bacterium Methylomonas Methanica; and the MLSS's composition will be 99.5% water and 0.5% bacterial-MLSS which is 90% bacterial mass by weight.

The bacterial content of the bacterial-MLSS is measured by taking a sample portion of the bacterial-MLSS. This sample has the relative composition measured as a loss in weight, by comparing the difference in weight which remains after drying at 103° C. to remove water, and removal of the volatiles at 600° C. The volatile fraction of the bacterial-MLSS, which is indicative of the bacterial weight, is typically 90% of the bacterial-MLSS. The bacterial-MLSS for the present invention is typically 5,000 mg/l or 0.5% by weight of the MLSS.

The typical time for the bacterial-MLSS to be replaced in the system by new bacteria is 8 to 10 days. So the volumetric throughput for liquids is measured in hours and the volumetric throughput for the bacterial mass is measured in days. For each pound of bacterial-MLSS, two pounds of methane gas (CH₄) will be oxidized by the methanotropic bacteria using four pounds of oxygen (O₂); and four pounds of methanol (CH₃OH) and two pounds of water will be produced. The efficiency of the process is measured by how closely the yield of methanol harvested from the process equals the completion of the process on a theoretical basis based on the above discussion.

Two distinct advantages of the present invention that leads to self sufficiency of the process which is the subject of the present invention are: (1) water is produced in the process; and (2) the oxidation of methane gas to methanol liquid is exothermic. There is substantial natural gas in remote locations or from oil wells that have been capped. These tend to be located in remote locations. The process has greater throughput at higher temperatures, so the heat produced can improve the yield of a facility or keep a facility operating at a useful level even in colder climates. These remote locations often have limited water available. Therefore, the present invention may be suitable for use in remote locations for the process generates water and thus the operation may require little to no make-up water after the initial charging of the process.

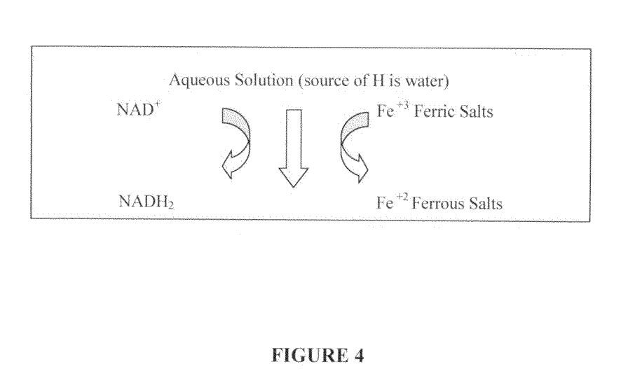

A first key point of this invention is controlling the environment for the bacteria, which enables control over their natural activity and production. The first reaction tank, after it has been mostly filled with the MLSS is sealed from the outside atmosphere, and connected to a controlled-atmosphere recirculation system for the duration of the first stage. The circulated atmosphere contains a stoichometric ratio for production of methanol of two pounds of oxygen per pound of methane, and its circulation starts by feeding a gas stream—in the preferred embodiment, by bubbling in from the bottom—into the first reaction tank, that gas stream being a 20:80, ratio by volume of methane-to-air. The diffused gas concentration is maintained throughout this first stage by recirculating the gas from the top of the first reaction tank back to the input, with this recirculated gas being returned to the proper ratio by scrubbing out carbon dioxide, condensing the water vapor, and adding methane or air as needed. (FIG. 4) By measuring the production of carbon dioxide it is possible to measure the progress and or rate of the process of generating the desired product methanol.

Oxygen is 21 percent by volume and 23.2 percent by weight of the Earth-normal atmosphere. To obtain a two-to-one molar ratio of oxygen to air for the preferred embodiment of this invention, matching the stoichometric ratio for the methanotropic bacteria's internal processing, assuming 100% methane in the methane gas source requires a 20% methane-to-air mixture for the first stage. This ratio can be adjusted depending on the purity of the methane gas source. For example, natural gas typically contains 75% methane, 20% ethane and 5% other hydrocarbons; and with those concentrations the natural-gas-to-air mixture will be 25%-natural-gas-to-75%-air. Gas from biological decomposition typically contains 65% methane with the balance being carbon dioxide; either this source is run first through a CO₂ scrubber, or the ratio is adjusted to 30.76% bio-methane mixture to 69.24% air. By increasing or decreasing the percentage mixture between the methane gas source and the normal atmosphere, the preferred methane-to-oxygen mixture can be continually piped into the first reaction tank.

The first stage thus uses an aerobic but abnormal atmospheric mixture of regular air and methane gas, that is kept below a non-flammable concentration. Contact between the volumetrically-measured bacterial mass and the air-methane mixture is controlled to ensure a continuing excess of methane over atmospheric norms that yet remains below the flammable limit of 5% (by weight of methane to the weight of oxygen). Continuing the reaction for the maximal productivity further requires removing unwanted metabolic products of water (H₂O) and carbon dioxide (CO₂) through a dehumidifier and a scrubber, respectively. The end point of this first, aerobic stage is determined by continuously measuring the oxidation reduction potential (ORP) of the bacterial mass with a pH meter that has a calamel electrode, that is an electrode made of mercurous chloride, a odorless solid with mercury as its principal component, at one end, and a platinum electrode at the other end, with the latter inserted into the reaction vessel slurry. The first stage is stopped when the ORP is minus 0.5 to 5 millivolts.

The probe from the pH meter is placed so that the measurements obtained are representative of the bulk of the mass in the first reaction tank. Both the first and the second reaction tank will be sealed and remain sealed after the contents placed therein to the desired filling point. The gas stream is recycled and the component produced in the process are removed continuously, with the gas stream supplemented with the mixture of methane or air as described herein to keep the concentrations at the desired level; and the air flow is maintained at sufficient level to keep the liquid, solid and gas components in contact by using the gas flow for mixing.

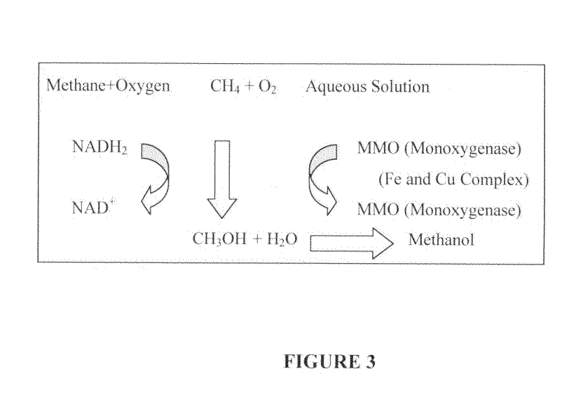

The methanotropic bacteria's methane-processing reaction in the first stage is shown in FIG. 3.

The reaction transforms one mole of methane gas to one mole of methanol using the methanotropic bacteria's evolved, organo-metallic MMO enzyme. MMO exists in the methanotropic bacteria as the result of untold eons of evolutionary search by trial and error for the most efficient transformation means to use an unusual resource—methane—found in certain evolutionary niches. This inherent efficiency of nature's biological processes, and their relative advantage over man-made chemical processing, is best exemplified by the firefly which can convert electrons to photons with an energy efficiency of 95%, while man after years of trying now can only attain a 35% conversion efficiency in chemoluminescence processing. The MMO reduces the energy need to make the oxidation of methane gas to methanol to occur as the reduction of NADH₂ to NAD⁺ occurs. The NADH₂ donates two hydrogen to the production of methanol.

In the preferred embodiment of the invention the biomass with water is contained and continuously mixed in the first reaction tank by diffusing the above gases in a process similar to the activated sludge process used in wastewater treatment. The operation is in a batch operation rather than the typical, waste-water plant, activated-sludge process that operates in a continuous operation The preferred embodiment uses a diffuser to maximize both the gas-to-liquid contact and the mixing of the biomass with the input gas.

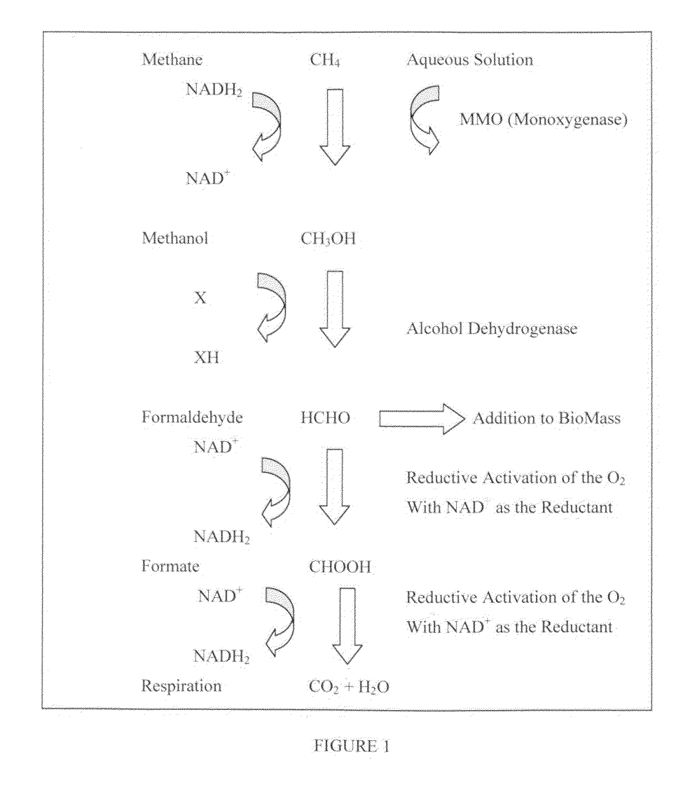

As noted, the end point for the first stage of methanol oxidation is reached when the ORP measures at minus 0.5 to 5 millivolts. In an alternative embodiment the first stage's end point is when the ORP measures at minus 1.0 to 1.5 millivolts. This end point allows the process to be interrupted in order to harvest the methanol-rich reaction liquor before the natural bacterial cycle continues; as shown in FIG. 1, if not interrupted the methanotropic bacteria will go on to produce first formaldehyde and then formate, followed by respiration, and the production of CO₂, H₂O and cell mass (reproduction).

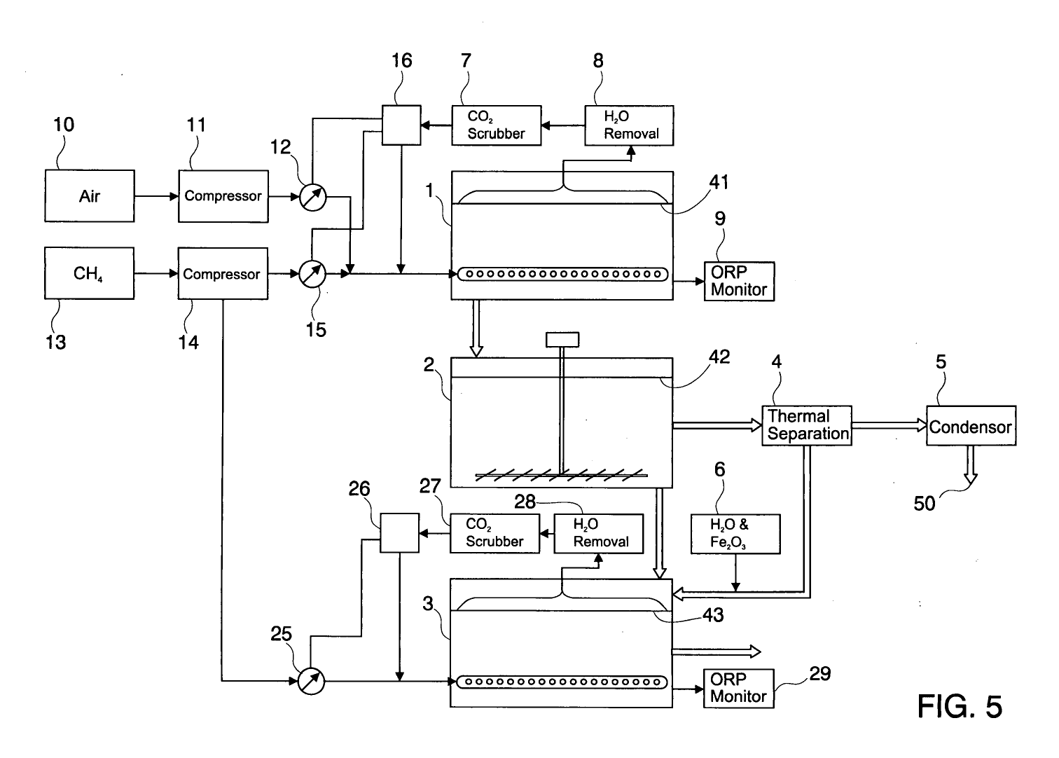

At this ORP-measured point the bacterial metabolic cycle will have produced the maximal harvestable surplus of methanol (CH₃OH), FIG. 1. In the first stage two pounds of methanol is produced in four hours for each pound of Mixed Liquor Suspended Solids. (FIG. 5, [50]).

Also, as the methanol production occurs, the color of the MLSS changes (from blue to green), which color change, with sampling, filtering and measuring well known to a person skilled in the art, can be used to monitor the process and signal the time to change to the next step, harvesting.

A second key aspect of this invention is the measured and timely interruption of the natural processes of the methanotropic bacteria, through alteration of the environmental constituents and constraints, to obtain the maximal production of the desired chemical products from the bacteria's ‘natural factories’. It is for this reason the input of air and methane gas is now halted and the reactive mass is sent to the clarifying means, which also operate at normal atmospheric and liquid-water conditions.

It is possible to interrupt the first stage's process at a later state, when the methanol has been processed into formaldehyde, since the number one use of methanol today is to produce formaldehyde.

In the preferred embodiment the clarifying means has its surface area sized to allow separation of the liquids and settling of the solids in 2 hours at 20° C., assuming a settling rate of 0.5 gallons per minute per square foot of surface loading. After the settling period, the liquid from the clarifying means is continuously drawn off for one hour and sent to thermal separation where the methanol is driven off as a gas by heating the liquid to or just above the boiling point of methanol, which is a lower temperature than the boiling point of water (64.6° C. vs. 100° C.). This methanol is processed in a water-cooled condenser to produce liquid methanol as a product. The water remaining after the methanol has been separated is sent to the second reaction tank. In an alternative embodiment the heat from the exothermic reaction of the first stage is drawn off and transferred to the clarifier through a temporary heat store to maximize cogenerative efficiency of the process.

Most of the carbon from the methane gas input to the first reaction tank that was consumed by the methanotropic bacteria has been removed by their metabolic processing and is now in and part of the methanol. However, under certain conditions the ‘solids’ portion of the MLSS left in the clarifying means can exceed the target of 5,000 mg/l or 0.5% w/v. Under these circumstances the excess solids will be disposed of after the required amount of solids are drawn off from the bottom of the clarifying means and placed into the second reaction tank, which like the first will have a MLSS for the present invention of 5,000 mg/l or 0.5% by weight and an gas/liquid interface after it has been filled with the MLSS.

In a further embodiment at least one reaction tank is used as a storage tank to provide storage for MLSS and/or condensed water from the thermal process that concentrates the methanol, thereby enabling flexibility of interfacing batch and the continuous unit operations and making these components available when the first reaction tank is available to be filled to start once more in aerobic mode to generate methanol.

The function of the second half of the cycle is regenerating the NAD⁺ within the methanotropic bacteria to NADH₂ so the biomass can be repeatedly used to convert methane into methanol. A key point to the selection and management of the methanotropic bacteria is the fact that they evolved alternative metabolic processes that operate differently depending on whether these bacteria are in an aerobic (ultimately, reproductive and respirative) environment, or an anaerobic (restorative) environment.

In the second stage the MLSS (and thus the methanotropic bacteria) will be maintained in an anaerobic environment and exposed to close to 100% methane gas. During this second stage no oxygen is to be present in the gas stream or in the second reaction tank. The absence of oxygen is monitored to keep the bacteria in the NADH₂ restorative cycle using the inorganics to replace the function of oxygen in the metabolic pathway of the microorganism; but this also serves to ensure that the methane-rich gaseous mixture remains above the Upper Flammability limit, which is a concentration of 15% methane w/w to oxygen.

The bacterial metabolic cycle, having been interrupted, now will be short-circuited to regenerate the NAD⁺ to NADH₂. These bacteria in anaerobic conditions engage in the conversion of ferric to ferrous salt and/or cupric to cuprous salts in the presence of decomposition of the excess biomass where the microorganisms are deprived of oxygen so they must turn to the metals and the transition of ferric to ferrous salts to support their metabolic process. The introduced iron and/or copper will serve a second purpose of enriching the iron-based or copper-based monooxygenase enzyme that principally drives the methane oxidation to methanol and allow the conversion to proceed at the very desirable conditions of ambient temperature and pressure.

There are changes in color in the visible light spectrum that occur and can be monitored associated with the reduction of NAD and the change of copper and iron salts from the ‘ferr-ic’ to the ‘ferr-ous’ forms. Ferric Oxide is rust or orange color and ferrous oxide is a black color.

In the preferred embodiment of the present invention water with ferric oxide (Fe₂O₃) dissolved into it is added to the second reaction tank with the MLSS already inside to fill the vessel to the design capacity. As with the first reaction tank, the second reaction tank is not filled entirely with the water+MLSS mixture; a gas/liquid interface level (surface) is left.

Next, the second reaction tank is sealed and the normal atmosphere is replaced by piping in methane gas until the atmosphere of the second reaction tank is as close to 100% methane gas as practical, and in any case has as close to zero content of oxygen as can be managed.

In this second stage the bacteria in the MLSS remaining after harvesting the methanol by clarification are once again contacted with methane gas, but this time in concentration and without the admission of normal air in order to maintain an anaerobic environment so the bacteria's principal reaction is shown in FIG. 4.

The methane gas concentration is maintained by recirculating the gas from the top of the second reaction tank back to its input, with this ‘output gas’ being returned to the proper ratio by scrubbing out carbon dioxide, condensing the water vapor and adding methane as needed. (FIG. 5)

The endpoint of this second stage, of bacterial enymatic reset, is when sufficient NADH₂ has been produced to once again support aerobic metabolization, which is measured by the oxidation reduction potential or ORP of the MLSS. The second stage's end point is when the ORP measures a positive 1 to 6 millivolts. In an alternative embodiment, the second stage's end point is when the ORP measures a positive 2 to 2.25 millivolts.

Once the change in the ORP has determined the end point, the contents of the second reaction tank now incorporate fully-reset bacteria. At this point, the cycle can be restarted. In a preferred embodiment, the contents of the second reaction tank will be drawn off, measured, and placed in proper proportion of bacterial-MLSS concentration in water to be placed back into the first reaction tank if the tank is ready to be charged or this content is to be stored until the reaction tank is ready to accept this charge.

Alternative further embodiments can be used without departing from the present invention where the bacterial-MLSS biomass is fixed to media such as set of one or more rotating discs of fixed rock, saddles or plastic packing, which media can be used in upward or downward flow patterns with the gas and the liquid starting and leaving the reaction tank in opposite directions as determined by gravitational flow or pumping, or by having the media (e.g. disc) alternatively dipping into the liquid followed by rising into the gas.

Alternative methods of mixing the gas and liquid can be used such as surface aeration/mixing devices or brush aerators in a race track formation, without departing from the present invention.

In the preferred embodiment each reaction tank is a 10,000 gallon concrete tank; the initial MLSS composition starts as a 99.5% water and a 0.5% bacterial-mixed liquor suspended solids, bacterial-MLSS (4,135 lb), of which the bacterial mass is 90% by weight of the volatile fraction thereof; and the reaction temperatures are operating at 20 degrees C. Each reaction tank is capable of being loaded with an equal quantity of biochemical oxygen demand (BOD) for the methanotropic bacteria, in terms of methane to be converted to methanol in four hours. The BOD of methane is 1 moles of oxygen (gram molecular weight of 32) per mole of methane (gram molecular weight of 16). On a weight basis there are two pounds of BOD to be processed by the activated sludge process for each pound of methane converted to methanol.

In a further embodiment of this invention the through-put of the manufacturing of Methanol is maximized by adopting sequences of operation that make best use of at least two reaction tanks, a clarifier and a storage means.

In a further embodiment of this invention, the reaction tanks will be logically swapped with the second reaction tank becoming the first reaction tank, and the former first reaction tank becoming the second reaction tank, and the process run through ‘backwards’ from the former second, now first, reaction tank to the clarifier to the former first, now second reaction tank. In a yet further embodiment of this invention where there are more than 2 reaction tanks attached to a single clarifying means, each tank when it is emptied joins a queue of ready tanks to be filled, and ordered, according to the stage at which the input MLSS will be operating (aerobic first or anaerobic second) and have the inputs and conditions governed accordingly.

The time for the two stages is approximately equal. At an operating temperature of 20° C. the time is approximately 4 hours for each stage. Stage One is an exothermal process producing heat so that by application of proper engineering it is possible to use the heat generated to raise the operating temperature of both modes, and thus increase throughput by reducing contact time as well as the use as heat for the thermal process used to concentrate the methanol. The stage processes are batch as it relates to the MLSS and liquid handling, and continuous (cyclic with monitored mixture, differentiated inputs and scrubbing) for the gases. The gas in Stage Two is the same methane-rich source gas used in Stage One without air. All tanks are covered and sealed from outside atmosphere during each stage. At the end of the 4 hours the gas flow to the first reaction tank is stopped and the first reaction tank is emptied to charge the second 10,000 gallon tank.

The bacteria are temperature sensitive. They are effective in producing methanol from methane operating in the temperature range of 10 to 40° C. For each 10° C. increase the bacterial activity rate doubles, so that at 30° C. the detention time is reduced to 2 hours and at 40° C. the detention time is one hour. The methane gas consumed is equal to one half the MLSS and the methanol produced is equal to the MLSS or 4,135 lbs, (5,200 gallons @0.8 specific gravity). The detention time for Stages One and Two are equal. If operation could be sustained at a temperature of 40° C. then daily production of methanol for a plant with two 10,000 gallon tanks would be 62,400 gallons (24/2*5,200 gallon). At a market price of $170 a metric ton in the U.S. ($0.42 a gallon) the value of the methanol produced is $26,562.

As an alternative embodiment, the timing of both the aerobic and anaerobic metabolization (stage one and stage two) can be governed through means other than ORP monitoring, such as spectral signals. Both NAD+ and NADH absorb strongly in the ultraviolet spectrum due to their adenine base. But while peak absorption of NAD+ is at 259 nanometers (nm), with an extinction coefficient of 16,900 M-1 cm-1; NADH, which also absorbs at higher wavelengths, has a second peak in UV absorption at 339 nm with an extinction coefficient of 6,220 M-1 cm-1. This difference in the ultraviolet absorption spectra between the oxidized and reduced forms of the co-enzymes at higher wavelengths allows the conversion between these forms to be monitored through measuring the amount of UV absorption at 340 nm using a spectrophotometer. Also, NAD+ and NADH also differ in their fluorescence qualities. NADH in solution has an emission peak at 460 nm and a fluorescence lifetime of 0.4 nanoseconds, but the oxidized form of the coenzyme does not fluoresce. Colorimetric observation, UV-absorptive measurement, or fluorescent quality monitoring, can all be used as alternative stage management means.

In a further embodiment there is only one reaction tank which is used alternately for both the first stage and the second stage; and in yet another further embodiment all of the methane gas, atomosphere, and recirculated gas inputs are switchably and separably controlled for each reaction tank by at least one methane-oxygen-concentration monitoring and metering valve.

It will be understood that the embodiments described herein are merely exemplary and that a person skilled in the art may make many variations and modifications without departing from the sphere and scope of the invention. All such variations and modifications are intended to be included in the scope of the invention as defined in the appended claims.

The claims stated herein should be read as including those steps and/or elements which are not necessary to the invention yet are in the prior art and are necessary to the overall function of that particular claim, and should be read as including, to the maximum extent permissible by law, known functional equivalents to the steps and/or elements disclosed in the specification, even though those functional equivalents are not exhaustively detailed herein. Accordingly, it is intended that the appended claims are interpreted as covering all alterations and modifications as fall within the true spirit and scope of the invention in light of the prior art.

Additionally, although claims have been formulated in this application to particular combinations, it should be understood that the scope of the disclosure of the present application also includes any single novel aspect or any novel combination of aspects disclosed herein, either explicitly or implicitly, whether or not it relates to the same invention as presently claimed in any claim and whether or not it mitigates any or all of the same technical problems as does the present invention. The applicant hereby give notice that new claims may be formulated to such features and/or combinations of such features during the prosecution of the present application or of any further application derived therefrom.

I claim:

1. A method for producing liquid methanol from methane gas at bio-normal temperatures and pressures, comprising:
first, filling a first reaction tank less than full, so as to leave a gas/liquid interface within it, with a mixed liquor suspended solids (‘MLSS’), said MLSS comprising 99.5% by weight water and 0.5% by weight a bacterial mixed liquor suspended solids concentration (‘bacterial-MLSS’) comprising 90% by weight of the volatile fraction thereof a bacterial mass of Type I bacterium Methylomonas Methanica;
sealing the first reaction tank against the outside atmosphere;
next, replacing the air left in the first reaction tank above the gas/liquid interface with a first gaseous mixture comprising by volume methane and oxygen in a 1:4 ratio;
establishing an operating temperature for the first reaction tank of 20° C.;
next, continuously circulating through the MLSS in the first reaction tank the first gaseous mixture comprising by volume methane and oxygen in a 1:4 ratio, said first gaseous mixture coming from a combination of a methane gas source, an atmosphere intake, and a recirculation pipeline from above the gas/liquid interface of the first reaction tank, with the recirculation pipeline incorporating a scrubber to remove excess CO₂ and a dehumidifier to remove excess water vapor, before connecting with the combination of the methane gas source and atmosphere intake, said combination being monitored and controlled by a methane gas concentration monitor, until a signal that the methanol production has peaked is produced;
producing a signal that the methanol production has peaked by monitoring the MLSS with an oxidation reduction potential monitor (‘ORP’) and obtaining a reading from the ORP in the range of minus 0.5 to positive 1.5 millivolts;
upon receiving a signal that the methanol production has peaked, stopping the circulation of the first gaseous mixture and transferring the MLSS from the first reaction tank into a clarifier, again leaving a gas/liquid surface in the clarifier;
next, establishing an operating temperature for the clarifier of 20° C.;
letting the solids in the MLSS settle in the clarifier for two hours;
then, running the liquid in the clarifier through a thermal separator for one hour, said thermal separator operating at or just above the boiling point of methanol, returning the liquid from the separator back to the clarifier and piping the distillate to a condenser, and running the distillate through the condenser to liquify the methanol;
thereafter, combining, in a second reaction tank, the solids and other contents from the clarifier, and the liquid from the thermal separator to which inorganic metallic salts taken from the set of ferric or cupric metallic salts have been added, and filling the second reaction tank less than full, so as to leave a gas/liquid interface within it;
sealing the second reaction tank against the outside atmosphere;
next, replacing the air left in the second reaction tank above the gas/liquid interface with a second gaseous mixture comprising as close as is practicable a 100% methane gas;
establishing an operating temperature for the second reaction tank of 20° C.;
next, continuously circulating through the MLSS in the second reaction tank the second gaseous mixture comprising as close as is practicable a 100% methane gas, said second gaseous mixture coming from a combination of a methane gas source and a recirculation pipeline from above the gas/liquid interface of the second reaction tank, with the recirculation pipeline incorporating a scrubber to remove excess CO₂ and a dehumidifier to remove excess water vapor, before connecting with the methane gas source, said combination being monitored and controlled by a methane gas concentration monitor, until a signal that the bacterial enyzmatic reset is peaking is produced;
producing a signal that the bacterial enyzmatic reset is peaking by monitoring the MLSS with an oxidation reduction potential monitor (ORP) and obtaining a reading from the ORP in the range of positive 2.0 to positive 6 millivolts;
upon receiving a signal that the bacterial enyzmatic reset is peaking, stopping the circulation of the second gaseous mixture and letting the solids in the MLSS settle for two hours; and,
restarting the cycle, establishing from the solids and liquids in the second reaction tank, to which water, solids, and bacterial mass are added as necessary, the MLSS comprising 99.5% by weight water and 0.5% by weight a bacterial mixed liquor suspended solids concentration (‘bacterial-MLSS’) comprising 90% by weight of the volatile fraction thereof a bacterial mass of Type I bacterium Methylomonas Methanica.

2. A method for producing liquid methanol from methane gas at bio-normal temperatures and pressures as set forth in claim 1, wherein the first reaction tank, clarifier, and second reaction tank are each of 10,000 gallon capacity, further comprising:
producing a signal that the methanol production has peaked upon the first receipt of any of a set of signals thereof, said set including the passage of four hours of continuous gas recirculation, obtaining a reading from the ORP in the range of minus 0.5 to positive 1.5 millivolts; and obtaining a spectral signal thereof; and,
producing a signal that the bacterial enyzmatic reset is peaking upon the first receipt of any of a set of signals thereof, said set including the passage of four hours of continuous gas recirculation, obtaining a reading from the ORP in the range of positive 2.0 to 6 millivolts, and obtaining a spectral signal thereof.

3. A method for producing liquid methanol from methane gas at bio-normal temperatures and pressures as set forth in claim 2 wherein the operating temperatures of 20° C. are increased to 30° C., and the time periods for producing a signal are reduced to 2 hours.

4. A method for producing liquid methanol from methane gas at bio-normal temperatures and pressures as set forth in claim 2 wherein the operating temperatures of 20° C. are increased to 40° C. and the time periods for producing a signal are reduced to 1 hour.

5. A method as set forth in claim 2, wherein the step of producing a signal that the methanol production has peaked upon the first receipt of any of a set of signals thereof includes for obtaining a spectral signal thereof, observing from a sample of the bacterial-MLSS UV absorption between 335 and 345 nm.

6. A method as set forth in claim 2, wherein the step of producing a signal that the methanol production has peaked upon the first receipt of any of a set of signals thereof includes for obtaining a spectral signal thereof, observing from a sample of the bacterial-MLSS a fluorescent emission in the range of 455 to 465 nm.

7. A method as set forth in claim 2, wherein the step producing a signal that the bacterial enyzmatic reset is peaking upon the first receipt of any of a set of signals thereof includes for obtaining a spectral signal thereof, observing from a sample of the bacterial-MLSS no fluorescent emission.

8. A method as set forth in claim 2, wherein the step of producing a signal that the methanol production has peaked upon the first receipt of any of a set of signals thereof includes for obtaining a spectral signal thereof, obtaining a measurement of the correct change of the MLSS from blue to green.

9. A method as set forth in claim 2, wherein the step of producing a signal that the bacterial enyzmatic reset is peaking upon the first receipt of any of a set of signals thereof includes for obtaining a spectral signal thereof, obtaining a measurement of the correct change of the MLSS from orange to black.

10) A method as set forth in claim 1, wherein a heat-storage-and-transfer means takes heat from the exothermic reaction in the first reaction tank and provides it to any of the clarifier, thermal separator, and second reaction tank, for cogenerative efficiency.

11) A method as set forth in claim 1, wherein the gas/liquid interaction between the recirculating gas and the MLSS is provided by any of a set of alternatives, said set including bubbling the gas in from the bottom, fixing the biomass to a media such as one or more rotating discs of fixed material and rotating the disc such that any portion of its surfaces alternates between gas and liquid, and using surface aeration and mixing devices operating at the gas/liquid interface.

12) A method as set forth in claim 1 wherein the gas/liquid interaction between the recirculating gas and the MLSS is provided by opposing continuous flows of liquid and gas maintained by gravitation, pumping means and recirculation channeling means.

13) A method as in claim 1, further comprising producing a signal that the methanol production has peaked upon obtaining a reading from the ORP in the range of minus 0.5 to positive 1.0 millivolts.

14) A method as in claim 1, further comprising producing a signal that the bacterial enyzmatic reset is peaking upon obtaining a reading from the ORP in the range of positive 2.0 to positive 2.25 millivolts.

15) A method as in claim 1, wherein the natural processes of the methanotropic bacteria will be differentially exploited and the methanol produced will be further processed by them into formaldehyde, further comprising:
continuously circulating through the MLSS in the first reaction tank the first gaseous mixture comprising by volume methane and oxygen in a 1:4 ratio, said first gaseous mixture coming from a combination of a methane gas source, an atmosphere intake, and a recirculation pipeline from above the gas/liquid interface of the first reaction tank, with the recirculation pipeline incorporating a scrubber to remove excess CO₂ and a dehumidifier to remove excess water vapor, before connecting with the combination of the methane gas source and atmosphere intake, said combination being monitored and controlled by a methane gas concentration monitor, until a signal that the formaldehyde production has peaked is produced;
upon receiving a signal that the formaldehyde production has peaked, stopping the circulation of the first gaseous mixture and transferring the MLSS from the first reaction tank into a clarifier, again leaving a gas/liquid surface in the clarifier;
next, establishing an operating temperature for the clarifier of 20° C.;
letting the solids in the MLSS settle in the clarifier for two hours;
separating out the formaldehyde from the MLSS, returning the remaining material to the clarifier;
preparing and filling the second reaction tank with the remaining material and such replacement liquids and solids in proportion as necessary;
sealing the second reaction tank against the outside atmosphere;
next, replacing the air left in the second reaction tank above the gas/liquid interface with a second gaseous mixture comprising as close as is practicable a 100% methane gas;
establishing an operating temperature for the second reaction tank of 20° C.;
next, continuously circulating through the MLSS in the second reaction tank the second gaseous mixture comprising as close as is practicable a 100% methane gas, said second gaseous mixture coming from a combination of a methane gas source and a recirculation pipeline from above the gas/liquid interface of the second reaction tank, with the recirculation pipeline incorporating a scrubber to remove excess CO₂ and a dehumidifier to remove excess water vapor, before connecting with the methane gas source, said combination being monitored and controlled by a methane gas concentration monitor, until a signal that the bacterial enyzmatic reset is peaking is produced;
producing a signal that the bacterial enyzmatic reset is peaking by monitoring the MLSS with an oxidation reduction potential monitor (ORP) and obtaining a reading from the ORP in the range of positive 2.0 to positive 6 millivolts;
upon receiving a signal that the bacterial enyzmatic reset is peaking, stopping the circulation of the second gaseous mixture and letting the solids in the MLSS settle for two hours; and,
restarting the cycle, establishing from the solids and liquids in the second reaction tank, to which water, solids, and bacterial mass are added as necessary, the MLSS comprising 99.5% by weight water and 0.5% by weight a bacterial mixed liquor suspended solids concentration (‘bacterial-MLSS’) comprising 90% by weight of the volatile fraction thereof a bacterial mass of Type I bacterium Methylomonas Methanica.

16. A method as in claim 1, further comprising at least one gas-mixture monitor for each operating vessel to measure the gaseous mixture above the gas/liquid interface to ensure that the methane-to-oxygen concentration remains either below the lower flammability limit (‘LFL’) or above the flammability limit (‘UFL’).

17. A method as in claim 2 wherein producing a signal that the methanol production has peaked upon the first receipt of any of a set of signals thereof, further includes in said set a signal based on observing the rate of production of carbon dioxide.

18. A method for producing liquid methanol from methane gas at bio-normal temperatures and pressures, comprising:
having a first and second reaction tank, each of which is connected via a bidirectional pipe to a clarifier, which in turn is connected by a bidirectional pipe to a thermal separator, which is connected to a condenser, which has a production outlet;
having also for each reaction tank a gas input separably and switchably connected to a methane-oxygen concentration monitoring metering valve;
having also for each reaction tank a gas recirculation pipe which at one end forms an output from the reaction tank that is above the maximum level of the gas/liquid interface during production, with the gas recirculation pipe connecting through means to remove CO2 and means to remove water vapor from gas recirculating through the gas recirculation pipe to another end separably and switchably connected to the methane-oxygen concentration monitoring and metering valve;
having also at least one methane gas source, connected to a compressor, connected to a valve, switchably connected to the methane-oxygen concentration monitoring and metering valve;
having also at least one air input, connected to a compressor, connected to a valve, switchably connected to the methane-oxygen concentration monitoring and metering valve;
having also at least two input gas lines running between the methane-oxygen concentration monitoring and metering valve and each reaction tank, such that the methane-oxygen concentration and monitoring and metering valve can separably monitor and control the volumetric ratio of methane to oxygen passing through each separate input line to each reaction tank;
first filling the first reaction tank less than full, so as to leave a gas/liquid interface within it, with a starting mixed liquor suspended solids (‘MLSS’), said starting MLSS comprising 99.5% by weight water and 0.5% by weight a bacterial mixed liquor suspended solids concentration (‘bacterial-MLSS’) further comprising 90% by weight of the volatile fraction of the bacterial-MLSS a bacterial mass of Type I bacterium Methylomonas Methanica;
sealing the first reaction tank against the outside atmosphere;
next, using the methane-oxygen concentration monitoring and metering valve to control the inputs from each of the methane gas source, air input, and gas recirculation pipe for the first reaction tank, replacing the air left in the first reaction tank above the gas/liquid interface with a first gaseous mixture comprising by volume methane and oxygen in a 1:4 ratio;
establishing an operating temperature for the first reaction tank of 20° C.;
next, continuously circulating through the MLSS in the first reaction tank the first gaseous mixture comprising by volume methane and oxygen in a 1:4 ratio, said first gaseous mixture coming from a combination of a methane gas source, an atmosphere intake, and a recirculation pipeline from above the gas/liquid interface of the first reaction tank, until a signal is produced that the methanol production has peaked in the MLSS in the first reaction tank;
producing a signal that the methanol production has peaked by any of the following set of observations: noting the passage of four hours of continuous gas recirculation, obtaining a reading from the ORP monitor in the range of minus 0.5 to positive 1.5 millivolts, observing from a sample of the bacterial-MLSS UV absorption between 335 and 345 nm, observing from a sample of the bacterial-MLSS a fluorescent emission in the range of 455 to 465 nm, or obtaining a measurement of the correct change of the MLSS from blue to green;
upon receiving a signal that the methanol production has peaked, stopping the circulation of the first gaseous mixture and transferring the MLSS from the first reaction tank into the clarifier, again leaving a gas/liquid surface in the clarifier;
next, establishing an operating temperature for the clarifier of 20° C.;
letting the solids in the MLSS settle in the clarifier for two hours;
then, running the liquid in the clarifier through the thermal separator for one hour, said thermal separator operating at or, just above the boiling point of methanol, returning throughout this period the liquid from the separator back to the clarifier and piping the distillate to the condenser, and running the distillate through the condenser to liquify the methanol and delivering the methanol to the production outlet;
thereafter, combining, in the second reaction tank, the solids and other contents from the clarifier, and the liquid from the thermal separator to which inorganic metallic salts taken from the set of ferric or cupric metallic salts have been added, and filling the second reaction tank less than full, so as to leave a gas/liquid interface within it;
sealing the second reaction tank against the outside atmosphere;
next, replacing the air left in the second reaction tank above the gas/liquid interface with a second gaseous mixture comprising as close as is practicable a 100% methane gas;
establishing an operating temperature for the second reaction tank of 20° C.;
next, continuously circulating through the MLSS in the second reaction tank the second gaseous mixture comprising as close as is practicable a 100% methane gas, said second gaseous mixture coming from a combination of a methane gas source and a recirculation pipeline from above the gas/liquid interface of the second reaction tank, said second gaseous mixture being monitored and controlled by methane-oxygen concentration monitoring and metering valve, until a signal that the bacterial enyzmatic reset is peaking is produced;
producing a signal that the bacterial enyzmatic reset is peaking by obtaining any of the following set of observations: noting the passage of four hours of continuous gas recirculation, monitoring the MLSS with the oxidation reduction potential monitor and obtaining a reading in the range of positive 2.0 to positive 6 millivolts, observing from a sample of the bacterial-MLSS no fluorescent emission, and obtaining a measurement of the correct change of the MLSS from orange to black;
upon receiving a signal that the bacterial enyzmatic reset is peaking, stopping the circulation of the second gaseous mixture and letting the solids in the MLSS settle for two hours; and,
restarting the cycle, using the solids and liquids in the second reaction tank to which water, solids, and bacterial-MLSS are added as necessary, a new cycle's starting MLSS comprising 99.5% by weight water and 0.5% by weight a bacterial mixed liquor suspended solids concentration (‘bacterial-MLSS’) comprising 90% by weight of the volatile fraction thereof a bacterial mass of Type I bacterium Methylomonas Methanica, switching the names and logical roles of the first and second reaction tank.

19. A method for producing liquid methanol from methane gas at bio-normal temperatures and pressures, comprising:
having a reaction tank connected via a bidirectional pipe to a clarifier, which in turn is connected by a bidirectional pipe to a thermal separator, which is further connected to a condenser, which has a production outlet;
having also for the reaction tank a gas input pipe, connected to a methane-oxygen-concentration monitoring and metering valve such that the methane-oxygen-concentration monitoring and metering valve can monitor and control the volumetric ratio of methane-to-oxygen passing through the gas input pipe;
having also for the reaction tank a gas recirculation pipe which at one end forms an output from the reaction tank that is above the maximum level of the gas/liquid interface during production, with the gas recirculation pipe connecting through means to remove CO2 and means to remove water vapor from gas recirculating through the gas recirculation pipe, to another end connected to the methane-oxygen-concentration monitoring and metering valve;
having also at least one methane gas source, connected to a compressor, connected to the methane-oxygen-concentration monitoring and metering valve;
having also at least one air input, connected to a compressor connected to the methane-oxygen-concentration monitoring and metering valve;
having an oxygen-reduction-potential monitor (‘ORP monitor’) connected to the MLSS in the reaction tank;
first filling the reaction tank less than full, so as to leave a gas/liquid interface within it, with a starting mixed liquor suspended solids (‘MLSS’), said starting MLSS comprising 99.5% by weight water and 0.5% by weight a bacterial mixed liquor suspended solids concentration (‘bacterial-MLSS’) further comprising 90% by weight of the volatile fraction of the bacterial-MLSS a bacterial mass of Type I bacterium Methylomonas Methanica;
sealing the reaction tank against the outside atmosphere;
next, using the methane-oxygen-concentration monitoring and metering valve to control the connections from each of the methane gas source, air input, and gas recirculation pipe, so as to replace the air left in the reaction tank above the gas/liquid interface with a first gaseous mixture comprising by volume methane and oxygen in a 1:4 ratio;
establishing an operating temperature for the reaction tank of 20° C.;
next, continuously circulating through the MLSS in the reaction tank the first gaseous mixture until a signal is produced that the methanol production in the MLSS in the reaction tank has peaked;
producing a signal that the methanol production has peaked by any of the following set of observations: noting the passage of four hours of continuous gas recirculation, obtaining a reading from the ORP monitor in the range of minus 0.5 to positive 1.5 millivolts, observing from a sample of the bacterial-MLSS UV absorption between 335 and 345 nm, observing from a sample of the bacterial-MLSS a fluorescent emission in the range of 455 to 465 nm, or obtaining a measurement of the correct change of the MLSS from blue to green;
upon receiving a signal that the methanol production has peaked, stopping the circulation of the first gaseous mixture and transferring the MLSS from the reaction tank into the clarifier, again leaving a gas/liquid surface in the clarifier;
next, establishing an operating temperature for the clarifier of 20° C.;
letting the solids in the MLSS settle in the clarifier for two hours;
then, running the liquid in the clarifier through the thermal separator for one hour, said thermal separator operating at or just above the boiling point of methanol, returning throughout this period the liquid from the separator back to the clarifier and piping the distillate to the condenser, and running the distillate through the condenser to liquify the methanol and delivering the methanol to the production outlet;
thereafter, combining in the reaction tank, the solids and other contents from the clarifier, and the liquid from the thermal separator to which inorganic metallic salts taken from the set of ferric or cupric metallic salts have been added, and filling the reaction tank less than full, so as to leave a gas/liquid interface within it;
sealing the reaction tank against the outside atmosphere;
next, replacing the air left in the reaction tank above the gas/liquid interface with a second gaseous mixture comprising as close as is practicable a 100% methane gas;
establishing an operating temperature for the second reaction tank of 20° C.;
next, continuously circulating through the MLSS in the reaction tank the second gaseous mixture comprising as close as is practicable a 100% methane gas, said second gaseous mixture coming from a combination of a methane gas source and a recirculation pipeline from above the gas/liquid interface of the reaction tank, said second gaseous mixture being monitored and controlled by the methane-oxygen-concentration monitoring and metering valve, until a signal that the bacterial enyzmatic reset is peaking is produced;
producing a signal that the bacterial enyzmatic reset is peaking by obtaining any of the following set of observations: noting the passage of four hours of continuous gas recirculation, monitoring the MLSS with the oxidation reduction potential monitor and obtaining a reading in the range of positive 2.0 to positive 6 millivolts, observing from a sample of the bacterial-MLSS no fluorescent emission, and obtaining a measurement of the correct change of the MLSS from orange to black;
upon receiving a signal that the bacterial enyzmatic reset is peaking, stopping the circulation of the second gaseous mixture and letting the solids in the MLSS settle for two hours; and, restarting the cycle, using the solids and liquids in the reaction tank adding water, solids, and bacterial-MLSS as necessary to prepare a new cycle's starting MLSS comprised once again of 99.5% by weight water and 0.5% by weight a bacterial mixed liquor suspended solids concentration (‘bacterial-MLSS’) comprising 90% by weight of the volatile fraction thereof a bacterial mass of Type I bacterium Methylomonas Methanica.
